(19) United States
(12) Patent Application Publication
(10) Pub. No.: US 2025/0235280 A1
(43) Pub. Date: Jul. 24, 2025

(54) FIXATION DEVICE FOR A THERAPY DEVICE

(71) Applicant: Medivation AG, Baden (CH)

(72) Inventors: Jan Stifter, Schneisingen AG (CH); Tobias Schwägli, Solothurn (CH)

(73) Assignee: Medivation AG, Baden (CH)

(21) Appl. No.: 19/035,379

(22) Filed: Jan. 23, 2025

(30) Foreign Application Priority Data
Jan. 24, 2024 (EP) 24153618.4

(51) Int. Cl. A61B 50/20; A61B 34/00; A61B 90/00

(52) U.S. Cl. CPC A61B 50/20; A61B 34/25; A61B 2090/0807; A61B 2090/0811

(57) ABSTRACT

A fixation device for fastening a therapy device in an opening arranged in a bone, comprising a sleeve element which is configured to be received in the opening, comprising a first sleeve element portion and a second sleeve element portion. The first end of the first sleeve element portion includes a grip element and the second end includes an extension. The second sleeve element portion includes a projection arrangement. The first sleeve element portion and the second sleeve element portion are rotatable about each other in such a way that the projection arrangement is arranged in a first position adjacent to the extension and in a second position is at a distance from the extension corresponding to at least 5% of the circumference.

### CROSS-REFERENCE TO RELATED APPLICATION

This application claims the benefit of European patent application no. EP 24153618.4, filed Jan. 24, 2024, the contents of which are hereby incorporated by reference in their entirety.

### BACKGROUND

The present invention relates to a fixation device for a therapy device, for example a surgical aid or a navigation aid, in particular a tracker for surgical navigation. The fixation device is configured to be attached to a bone.

### STATE OF THE ART

Fixation devices for attaching tracking devices to bone for surgical navigation are required and used in many existing applications.

The attachment of surgical navigation trackers using a skull clamp is described for several neurological procedures. For example, U.S. Pat. No. 10,433,763 B2 describes a system in which a tracker is attached to a skull clamp. Other systems utilize a cranial frame with screws and pins that attach trackers to the skull (e.g., Brainlab's “Reference Base Skull” instrument). The pre-known fixation devices allow for rigid attachment of trackers to the bone outside the area of surgical intervention and require additional incisions and drilling or the placement of screws and/or staples.

In most cranial procedures, access to the brain is created by drilling one or more openings, also known as burr holes. These openings are typically in the range of 5 to 20 mm, inclusive, in diameter and allow access to the brain through a portal. If larger portions of the skull need to be removed, multiple openings are provided and then connected to a saw. Solutions for attaching instruments or targeting devices to such openings are described, for example, in the documents U.S. Pat. Nos. 9,675,783 B2, 10,307,218 B2 or 10,905,497 B2.

The object of the invention is to provide a fixation device by means of which a therapy device can be mounted in an opening in the skull bone of a patient, wherein the mounting device makes it possible to subsequently attach or remove a therapy device without removing the fixation device.

### SUMMARY OF THE INVENTION

When the term “for example” is used in the following description, this term refers to examples of embodiments and/or variants, which is not necessarily to be understood as a more preferred application of the teaching of the invention. Similarly, the terms “preferably”, “preferred” are to be understood as referring to an example from a set of embodiments and/or variants, which is not necessarily to be understood as a preferred application of the teaching of the invention. Accordingly, the terms “for example”, “preferably” or “preferred” may refer to a plurality of examples of embodiments and/or variants.

The following detailed description contains various embodiments of the fixation device according to the invention. The description of a particular fixation device is to be considered exemplary only. In the description and claims, the terms “including”, “comprising”, “containing” are interpreted as “including, but not limited to”.

A fixation device according to the invention for fastening a therapy device in an opening arranged in a bone contains a sleeve element which is configured to be received in the opening. The sleeve element contains a first sleeve element portion and a second sleeve element portion. The first sleeve element portion surrounds the second sleeve element portion in the installed state. The first sleeve element portion is configured as a cylindrical hollow body with a longitudinal axis. The first end of the first sleeve element portion includes a grip element. The second end of the first sleeve element portion includes an extension. The second sleeve element portion is configured as a cylindrical hollow body with a longitudinal axis. The second sleeve element portion contains a projection arrangement. The first sleeve element portion and the second sleeve element portion are rotatable about each other in such a way that the projection arrangement is arranged in a first position adjacent to the extension and the projection arrangement is arranged at a distance from the extension in a second position.

In particular, the distance can correspond to at least 5% of the circumference. The greater the distance between the projection arrangement and the extension, the greater the space required for the fixation device in the opening. The fixation device can thus be used for an opening whose diameter deviates from the nominal diameter.

This deviation can, in particular, be within a range of up to one millimeter if the diameter of the opening is in the range from 10 mm up to and including 15 mm. Depending on the actual diameter of the opening, the distance between the projection arrangement and the recess can be adjusted accordingly. The fixation device is thus adjustable so that inaccuracies in the production of the opening can be compensated for by the fixation device. In other words, the angle of rotation through which the grip element with a holding element connected to the second sleeve element portion can be enclosed can be adjustable. The grip element can in particular be turned relative to the holding element up to a stop of the holding element. The stop of the holding element is arranged in particular such that the extension and the projection arrangement assume the maximum possible distance from one another. In particular, the maximum possible distance can correspond to almost 50% of the circumference. In particular, the projection arrangement can be arranged in the second position opposite the extension.

The invention relates to a fixation device for fastening a therapy device, for example a surgical aid, in particular an instrument, for example a surgical tracker, which is configured to be attached to a bone, for example a skull bone. The fixation device is specially configured to be attached in an opening in which, for example, a surgical procedure can be performed, for example a drill hole in the skull bone. The fixation device contains an opening so that surgical navigation of the therapy device is also possible directly through the fixation device. The fixation device placed in the opening of the bone does not require any additional incision or other types of fixation, such as screws or staples.

According to an embodiment, the first sleeve element portion contains a first flange and a second flange. The first and second flanges enable additional fixation and stabilization of the fixation device. Such fixation can be realized in various ways, in particular the second flange can partially or completely rest on the opening. In particular, the second flange can have the function of a collar. The collar prevents the fixation device from being inserted too far into the opening and damaging underlying tissue. In addition, the collar can provide additional stability. The collar allows the fixation device to be positioned in a defined manner in the opening when installed. In particular, a skull bone may have a harder bony part with a thickness of a few millimeters. The second flange can have at least one tongue with an opening for receiving a fastener, for example a screw, according to an embodiment not shown. The screw can be used to ensure improved and permanent fixation of the fixation device.

The first flange and the second flange are arranged at a distance from each other according to an embodiment. The distance between the first and the second flange corresponds to the thickness of a layer of tissue covering the bone. In particular, the distance between the first flange and the second flange can be at least 5 mm. The first flange can rest on the layer of tissue. In particular, the grip element can also rest on the layer of tissue, so that operating the grip element cannot result in injury to the layer of tissue.

Additional profiling at the contact surfaces, in particular of the projection arrangement and/or the extension, can enable a more stable fixation of the fixation device to the bone. Contact surfaces are those surfaces of the fixation device that are in contact with the bone when installed. The profiling can comprise at least one element from the group consisting of ribs, grooves, teeth, spikes, points, cutting elements or threads.

According to an embodiment, a press-fit design is used in which the cross-section of the sleeve element is slightly larger than the cross-section of the opening, at least at the contact points or contact surfaces of the inner wall of the opening, so that the sleeve element is pressed against the inner wall at least at the contact points or contact surfaces. A press-fit design can also be realized by means of an additional assembly or disassembly aid, such as an inserter or a retractor. The assembly aid is used when the fixation device is inserted into the opening. The disassembly aid is used when the fixation device is removed.

According to an embodiment, the extension extends beyond the second flange in the direction of the longitudinal axis of the cylindrical hollow body of the first sleeve element portion.

According to an embodiment, the extension extends over part of a circumference of the first sleeve element portion. A fixation device having an extension that extends only over a small portion of the circumference of the sleeve element allows for a particularly easy insertion of the sleeve element into the opening.

According to an embodiment, the extension has a segment length that is in the range of 10% to 40% (inclusive) of the circumference of the cylindrical hollow body. The segment length of the extension allows the fixation device to be adapted to the diameter of the opening. The diameter of the opening in the bone cannot be manufactured exactly to the corresponding nominal dimension, since the bone is not homogeneous and, as an organic material, is not always of the same consistency. Therefore, the effective diameter is subject to a deviation in the range of approximately 1 mm when the nominal diameter is in the range of 10 to 15 mm, inclusive. The fixation device according to the invention thus enables a tolerance compensation. Depending on the effective diameter of the opening, the extension can be moved away from the projection arrangement just enough until a noticeable resistance is felt. When the grip element can no longer be turned without application of considerable force, the fixation device is fixed in the opening.

According to an embodiment, the extension has an axial length that is in the range of 5% to 50% inclusive of the distance measured between the first end and an end in the direction of a longitudinal axis of the first sleeve element portion.

According to an embodiment, the extension has a profiling on the outside. The profiling can enable the extension to engage in the inner wall of the opening, whereby an improved fixing of the fixation device in the opening is achievable.

According to an embodiment, the projection arrangement includes a first projection and a second projection. Each of the first and second projections has, in particular, a segment length that is in the range of 1% to a maximum of 10% of the circumference. The distance between the first projection and the second projection corresponds to at least the segment length of one of the first or second projections.

According to an embodiment, the projection arrangement includes a profiling. More particularly, the outer surface of the projection arrangement includes the profiling. The profiling can include at least one element selected from the group consisting of ribs, grooves, teeth, studs, spikes, cutting elements or threads.

According to an embodiment, the second sleeve element portion has an outer side that contains at least one recess. The recess allows the holding element to be precisely positioned on the second sleeve element portion. In particular, the holding element can be attached to the second sleeve element portion by means of a welded connection, an adhesive connection or a press fit in such a way that any relative movement between the second sleeve element portion and the holding element is prevented. In particular, the second sleeve element portion and the holding element form a rigid connection with each other. According to an embodiment, the holding element and the second sleeve element portion can consist of a single piece.

According to an embodiment, the second sleeve element portion includes a holding element. In particular, the holding element is attached to a first end of the second sleeve element portion. The holding element can be configured to receive a holding device. The holding device can be fixedly attached to the holding element or can be removable from the holding element. For example, the holding device can be configured to receive a tracker. The tracker can either be rigidly connected to the fixation device or designed so that it can be detached from the holding device and reattached without changing its spatial position in relation to the holding device. The fixation device is configured to provide a stable, fixedly located fixation when the tracker is removed from or reattached to the holding device.

The invention can be used in combination with a navigation system in which a tracker must be rigidly attached to the bone. The navigation system can in particular be a surgical navigation system. A system comprising the fixation device and a surgical navigation system can preferably be used for head surgery, such as shunt placement, cranial/neuro navigation, biopsies, ENT navigation or endoscopic navigation.

The invention also relates to a fixation device for a therapy device, for example a surgical aid, for example a navigation aid, in particular for a tracker. The fixation device is particularly designed to be rigidly fixed in an opening in the bone, which opening can particularly comprise a pre-drilled hole. The fixation device particularly allows a rigid attachment of a holding device, for example for a tracker on a bone for surgical navigation.

An advantage of the fixation device according to the invention is that no additional fixation element, such as a staple or a screw, is required. The fixation device can be attached directly at the site of the intervention, which allows for a simpler and less invasive procedure. The fixation device can be configured in such a way that access to structures below the opening is possible. The opening is thus not only used to hold the fixation device, but also to fasten the therapy device, in particular the surgical aid. According to the present embodiment, the fixation device also contains an access opening to tissue located within the body part for therapeutic treatment. In particular, it is advantageous if the tissue below the opening remains accessible for a subsequent surgical procedure after the fixation device has been installed. Such a surgical procedure can comprise the positioning of a stylet, the placement of a catheter, a cable or a probe through the opening in the fixation device when it is inserted in the opening. The therapy devices required for such a surgical procedure usually have a diameter of less than 3 mm. In particular, the inner diameter of the second sleeve element can be at least 3 mm. According to this embodiment of a therapy device, the fixation device inserted into the opening can allow access to the desired tissue or the desired anatomical area.

The fixation device in particular comprises a sleeve element which is advantageously configured to be correspondingly thin-walled and as short as possible in order to be able to be fastened in a thin bone and to leave space for a therapy device that can be passed through the fixation device inserted into the opening. A thin bone is understood to be a bone with little wall thickness, for example a skull bone.

Another advantageous feature of the fixation device is the option of being able to attach or remove the fixation device at a later date if a therapy device, such as a mandrin, catheter, cable or probe, is already located in an existing opening or has to remain in place after an intervention.

According to an embodiment, the first sleeve element portion has a longitudinal slot. In particular, according to this embodiment, the longitudinal slot extends from a first end of the first sleeve element portion to a second end of the first sleeve element portion. In particular, the longitudinal slot can have a slot width that is greater than the diameter of the therapy device.

According to an embodiment, the second sleeve element portion has a longitudinal slot. According to this embodiment, the longitudinal slot extends in particular from a first end of the second sleeve element portion to a second end of the second sleeve element portion.

In particular, the longitudinal slot of the first sleeve element portion and the longitudinal slot of the second sleeve element portion can be larger or may be enlarged, so that the slot width of the longitudinal slots is larger than the diameter of the therapy device. If necessary, the fixation device can be attached in the opening or removed from the opening without having to remove the therapy device. The longitudinal slot of the first sleeve element portion and the longitudinal slot of the second sleeve element portion are arranged one above the other for this purpose. The sleeve element, in particular in the form of the first sleeve element portion and the second sleeve element portion, thus extends only over part of the circumference of the opening. In other words, the sleeve element thus does not occupy the full circumference of the opening.

The fixation device can be used in many surgical procedures, e.g., in neurosurgery, for the placement of shunts, in ENT and in dental surgery, particularly when the guidance of a therapy device relative to an opening in the bone is desired. The surgical procedure can be performed either through the opening itself or in the vicinity of the opening in any direction.

A holding element attached to the fixation device can, for example, hold one or more parts of a navigation system. The navigation system can comprise at least one tracker. The tracker can, for example, be configured as an active or passive optical marker that can be used in combination with a single camera or a stereo camera system. A marker can also be configured as a sensor for electromagnetic navigation. The tracker or trackers can also be integrated or inside-out trackers, or trackers that use shadow imaging technology or markerless navigation technologies.

Using a tracking system, the relative position of the therapy device to the anatomy can be determined according to the position of the tracker attached to the bone relative to the therapy device, which is also equipped with a tracker. The tracking system is part of a navigation system, which requires a computing unit for calculating the spatial relationship of the tracker to its counterpart in addition to the tracking system. For example, a tracker attached to the fixation device can be tracked by a camera. Such a tracker is also referred to as a marker and can be passive or active, wherein an active tracker can be battery-powered or wired.

The fixation device can be used to mount a tracker that contains a camera or is formed as a camera. The camera is part of a navigation system, whereby the spatial position of tracker and camera can be calculated by means of the navigation system. In particular, miniaturized and correspondingly lightweight cameras can be used if a tracking system based on shadow imaging technology is employed. According to an embodiment, a camera used in a tracking system based on shadow imaging technology contains an optical sensor covered by an optical grid. The navigation system can calculate the position of an active optical marker, which is equipped with several LEDs, for example, on the basis of the shadow cast by the optical grid onto the image sensor.

The fixation device can be used to attach a fiducial marker in combination with the tracker or to enable its attachment. This enables image-based surgical procedures in which the mounted or integrated fiducial marker is used to register an image such as a C-arm, ISO-3D C-arm or CT on the navigation coordinate frame.

The described fixation device can advantageously be used in combination with a navigation system. The navigation system comprises the tracking system, whereby the position of at least one therapy device with a tracker in relation to the fixation device can also be measured by means of the navigation system, a computing unit for calculating the position of the therapy device on the basis of the measured position of the tracker, and a display unit, in particular for displaying navigation information for the surgeon. Instead of a monitor, an augmented reality headset or a display unit can be used to display the navigation information, which allows the navigation information to be overlaid on the surgical field.

The navigation system implements a surgical navigation workflow that may include the steps of preoperative imaging and planning, the import and intraoperative registration of the plan, and then the performance of the procedure under the guidance of the surgeon.

The fixation device according to the previous examples can be used in the sterile area. Accordingly, the fixation device can be configured as a disposable or reusable fixation device.

### BRIEF DESCRIPTION OF THE DRAWINGS

The fixation device according to the invention is shown below with reference to an example. It is shown in

FIG. 1 a fixation device according to an exemplary embodiment of the invention in the installed state,

FIG. 2a a fixation device according to a first embodiment in an exploded view in a first position,

FIG. 2b the fixation device according to FIG. 2a in the assembled state in the first position,

FIG. 3a an exploded view of the fixation device without the holding device,

FIG. 3b a view of the fixation device according to FIG. 2b in a second position,

FIG. 4a a perspective view of the fixation device with the holding element in a first position,

FIG. 4b a further perspective view of the fixation device with the holding element in the first position,

FIG. 5a a perspective view of the fixation device with the holding element in a second position,

FIG. 5b a further perspective view of the fixation device with the holding element in the second position,

FIG. 6a a radial section through the fixation device according to FIG. 2a in the first position,

FIG. 6b a radial section through the fixation device according to FIG. 3b in the second position,

FIG. 7a a perspective view of a fixation device according to a second embodiment with the holding element in a first position,

FIG. 7b a further perspective view of the fixation device according to FIG. 7a with the holding element in the first position,

FIG. 8a a perspective view of the fixation device according to the second embodiment with the holding element in the second position,

FIG. 8b a further perspective view of the fixation device with the holding element in the second position,

FIG. 9 an example of the use of the fixation device according to one of the embodiments.

### DETAILED DESCRIPTION

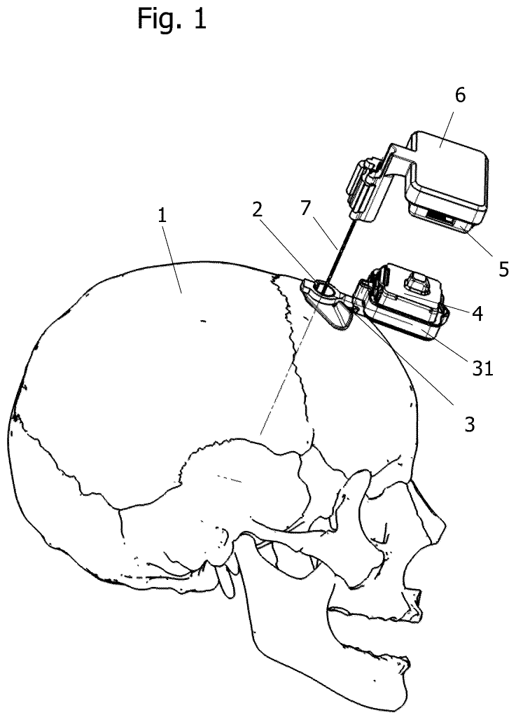

FIG. 1 shows an example of a fixation device 2 for a tracker arrangement and for guiding a therapy device 6, in particular a surgical instrument, for example a mandrin 7 for shunt placement, through an opening 9 in a skull bone 1 that is not visible in FIG. 1. This opening 9 can be realized in particular as a borehole. The fixation device 2 is received in the opening in such a way that, when installed, it projects at most insignificantly beyond the opening 9.

The tracker arrangement includes a first tracker 4 and a second tracker 5. The first tracker 4 can in particular include an optical marker or be configured as an optical marker. The second tracker 5 can include a camera. The first tracker 4 is rigidly attached to the skull bone 1 by means of the fixation device 2, so that no additional incisions or fixations on the skull bone 1 need to be provided.

The first tracker 4 is mounted on a holding element 3, which can be connected to the fixation device 2. The holding element 3 can thus be removed from the fixation device 2. According to the present embodiment, the second tracker 5 is connected to the therapy device 6. The therapy device 6 is thus connected to the second tracker 5 according to the present embodiment and can be navigated with respect to the first tracker 4 received in the holding element 3. Since the holding element 3 can be attached to the fixation device 2 in a defined position relative to the opening, the position of the first tracker 4 on the skull bone 1 and the soft tissue below the skull bone 1 can be determined via the hereby defined position of the first tracker 4.

In the present example, the position of the first tracker 4 relative to the second tracker 5 is determined. If the first tracker 4 contains an optical marker, its 6DOF position can be measured relative to the second tracker 5 and its camera. Alternatively, the second tracker 5 can also contain an optical marker, whereby an external camera not belonging to the tracker arrangement can be used to determine the 6DOF position of the first tracker 4 relative to the second tracker 5. According to a further embodiment, the therapy device 6 and the fixation device 2 can include optical markers so that the 6DOF position of the fixation device 2 and the therapy device 6 can be determined.

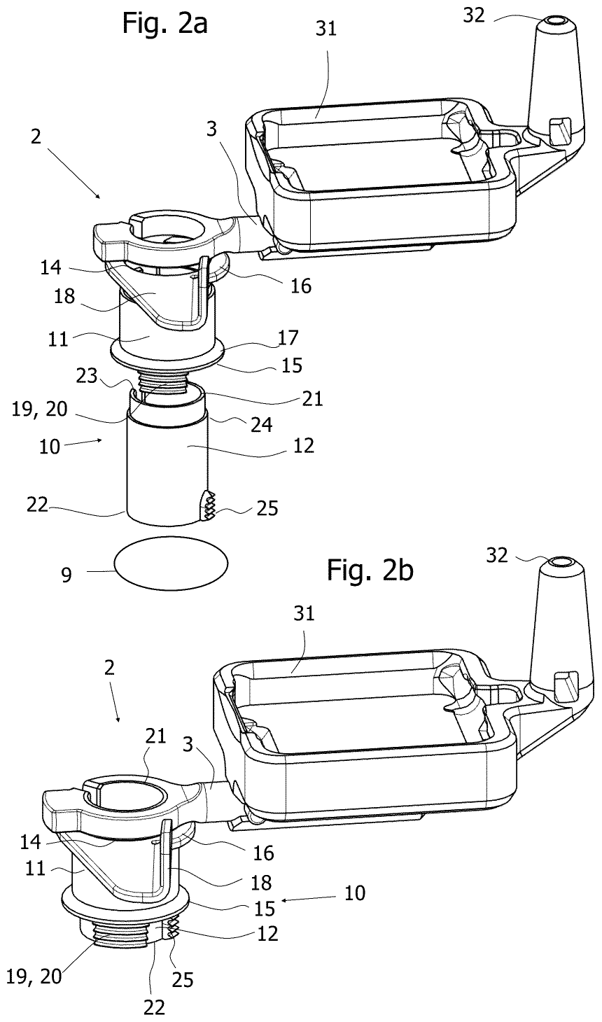

FIG. 2a shows the fixation device 2, which is to be received in the opening 9. The fixation device 2 includes a sleeve element 10, which is configured to be received in the opening 9. The sleeve element 10 includes a first sleeve element portion 11 and a second sleeve element portion 12. FIG. 2a shows an exploded view of the fixation device 2, and in FIG. 2b the fixation device 2 is shown in the assembled state. In the assembled state, the first sleeve element portion 11 is pushed over the second sleeve element portion 12. The first sleeve element portion 11 is rotatable relative to the second sleeve element portion 12. In particular, the first sleeve element portion 11 can be rotated around the second sleeve element portion 12.

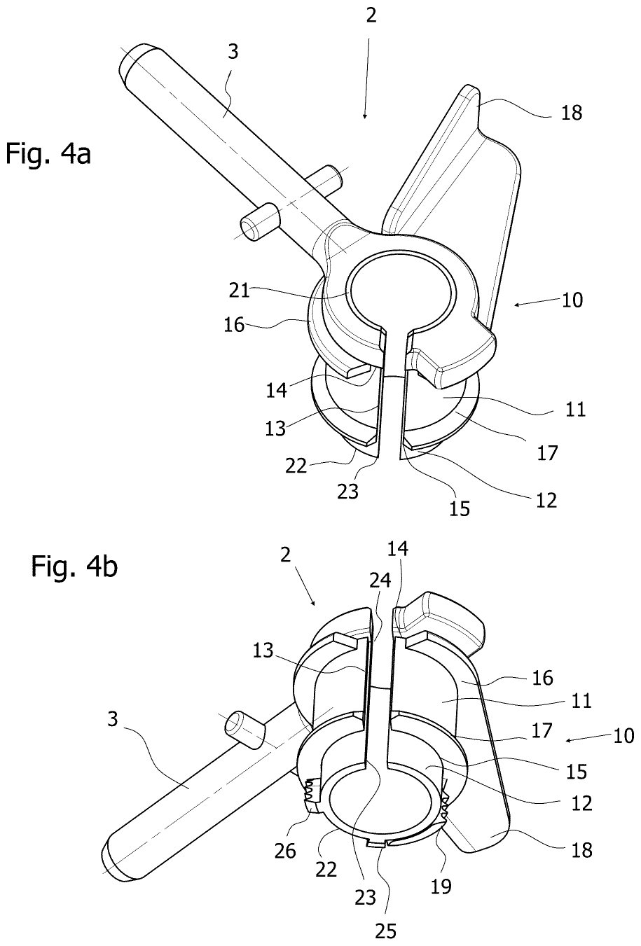

The first sleeve element portion 11 is configured as a cylindrical hollow body with a longitudinal slot 13, as shown in FIG. 4a or FIG. 4b. The longitudinal slot 13 extends from a first end 14 of the first sleeve element portion 11 to a second end 15 of the first sleeve element portion 11. The first end 14 of the first sleeve element portion 11 comprises a first flange 16 and a grip element 18. The second end 15 comprises a second flange 17 and an extension 19 that projects beyond the second flange 17 in the direction of the longitudinal axis of the cylindrical hollow body. The extension 19 extends over part of the circumference of the first sleeve element portion 11. According to the present example, a shell-shaped projection is formed by the extension 19. In particular, the extension 19 has a radius of curvature that corresponds to the inner diameter of the cylindrical hollow body. In the present example, the extension 19 has a segment length that is in the range from 10% to 40% inclusive of the circumference of the cylindrical hollow body. The extension 19 has an axial length which, according to the present example, is in the range of 5% to 50% of the distance measured between the first flange 16 and the second flange 17 in the direction of the longitudinal axis.

The extension 19 has a profiling 20 on the outside, according to the present example. The profiling can comprise at least one element from the group consisting of ribs, grooves, teeth, spikes, points, cutting elements or threads. In particular, the profiling can comprise ribs or grooves. The ribs or grooves extend in a substantially radial direction according to the present embodiment. The profiling creates a rough surface that provides an improved hold in the opening 9. The profiling increases the friction between the inner wall of the opening 9 and the first sleeve element portion 11, so that the first sleeve element portion 11 can wedge itself in the opening 9 when installed. Alternatively, the profiling can be configured in such a way that it can engage with the inner wall of the opening.

The second sleeve element portion 12 is configured as a cylindrical hollow body, which has a longitudinal slot 23, as shown in FIG. 4a or FIG. 4b. The longitudinal slot 23 extends from a first end 21 of the second sleeve element portion 12 to a second end 22 of the sleeve element portion 12. According to the present embodiment, the second sleeve element portion 12 has an inside with a smooth surface. In particular, the inside of the second sleeve element portion 12 contains no unevenness, protrusions or cross-sectional changes. A therapy device, such as a surgical instrument, a measuring device, a cannula or a tube, can be passed through the interior formed by the inside of the second sleeve component 12. According to the present embodiment, the second sleeve component 12 has an outer side that has at least one recess 24. According to the present embodiment, the second sleeve element portion 12 has a projection arrangement comprising at least one projection 25, 26. The recess 24 is closer to the first end 21 than to the second end 22. The recess 24 serves to support the holding element 3, which, in the installed state, as shown in FIG. 2b, FIG. 4a or FIG. 4b, rests on the recess 24. The holding element 3 is firmly connected to the second sleeve element portion 12, for example glued, welded or screwed. Alternatively, the holding element 3 and the second sleeve element portion 12 can be configured as a single component.

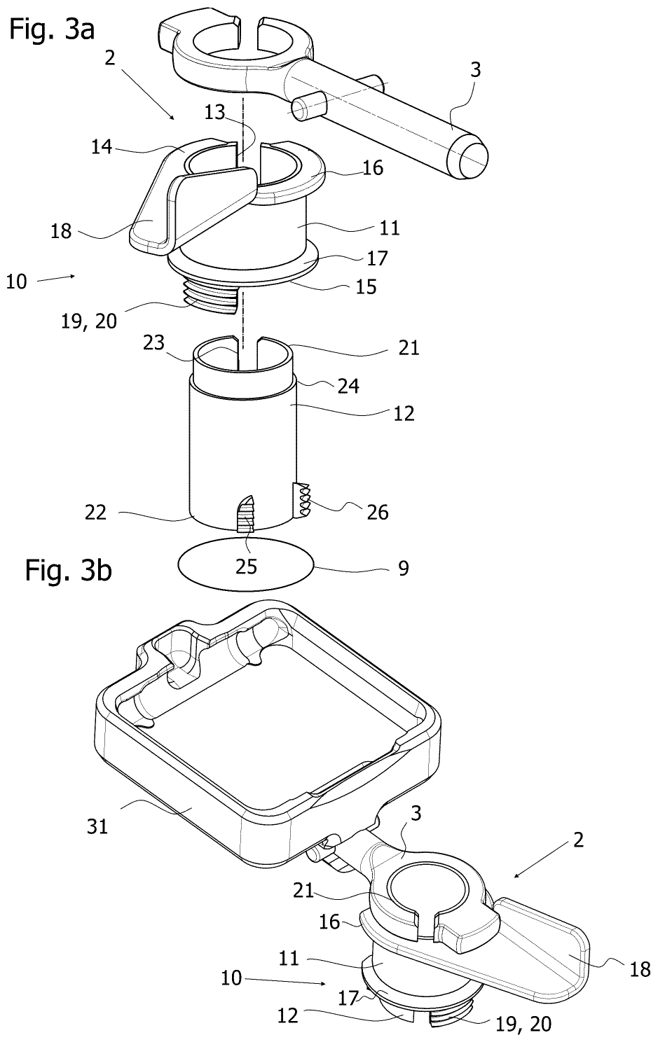

According to the present embodiment, the second sleeve element portion 12 has a first projection 25 and a second projection 26. In FIG. 2a and FIG. 2b, only the first projection 25 is visible, while in FIG. 3a, the first projection 25 and the second projection 26 are shown. At least one of the first projections 25 and the second projections 26 can have a profiling, for example in the form of ribs, grooves, teeth, spikes or threads. When installed, the first and second projections 25, 26 are in contact with the inner wall of the opening 9 and can form a contact surface with the inner wall of the opening 9. By means of a profiling, a more stable fixing of the fixation device 2 in the opening 9, for example in the borehole in the body part, can be achieved at the contact surfaces.

In particular, the profiling can be configured as a plurality of grooves or ribs that run in the radial direction. The segment length of the corresponding first or second projection 25, 26 can, in particular, be a maximum of 10% of the circumference of the second sleeve element portion 12 at its second end 22. The corresponding first or second projection 25, 26 can be a maximum of 30% of the total length of the second sleeve element portion 12 in the axial direction. The total length refers to the distance in the axial direction from the first end 21 to the second end 22.

Since, according to the present embodiment, both the first sleeve element portion 11 and the second sleeve element portion 12 each have a longitudinal slot 13, 23, it is easily possible to compress the first and second sleeve element portions 11, 12 by means of a tool in order to facilitate the introduction of the fixation device 2 into the opening 9. Such a longitudinal slot 13, 23 is also advantageous for later removal of the fixation device 2 from the opening 9, even if a therapy device 6 inserted through the opening 9 and through the fixation device 2 is to remain in place. An introducer or forceps can be used to compress the fixation device 2.

The holding element 3 can be configured to hold a holding device 31. The holding device 31 can be configured, for example, to hold a tracker, in particular the first tracker 4 shown in FIG. 1. The holding device 31 can be removable from the holding element 3 and is configured in such a way that it can be removed at a predetermined spatial position and can also be reattached in this predetermined spatial position. According to the present embodiment, the holding device 31 includes a calibration element 32. The calibration element 32 is configured to enable calibration or registration of a therapy device, for example the therapy device 6 shown in FIG. 1. For example, the calibration element 32 can comprise a calibration opening or a calibration channel into which a counterpart of the therapy device, designed as a tip element, can be inserted.

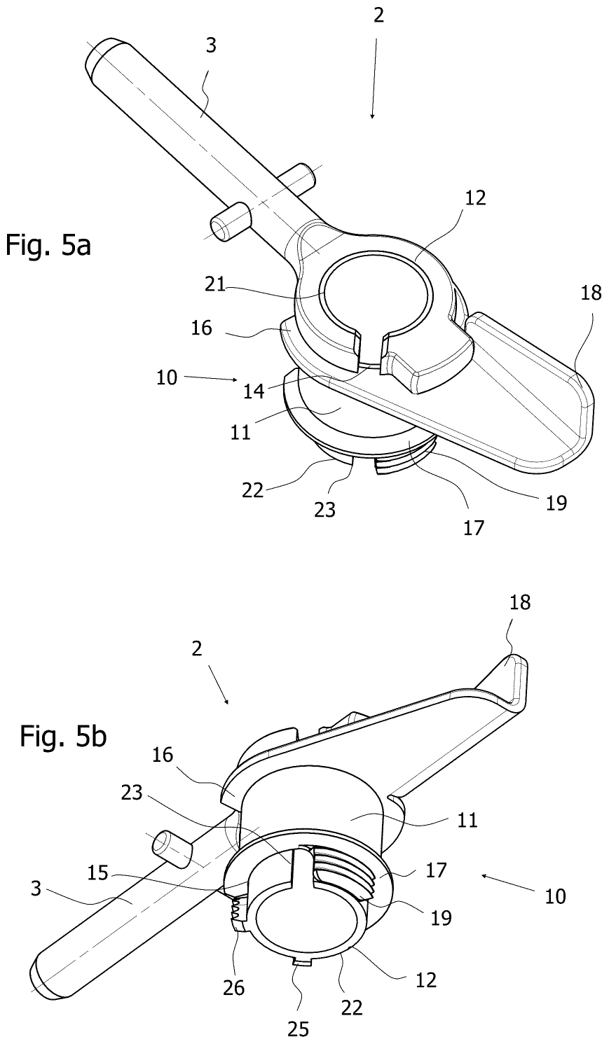

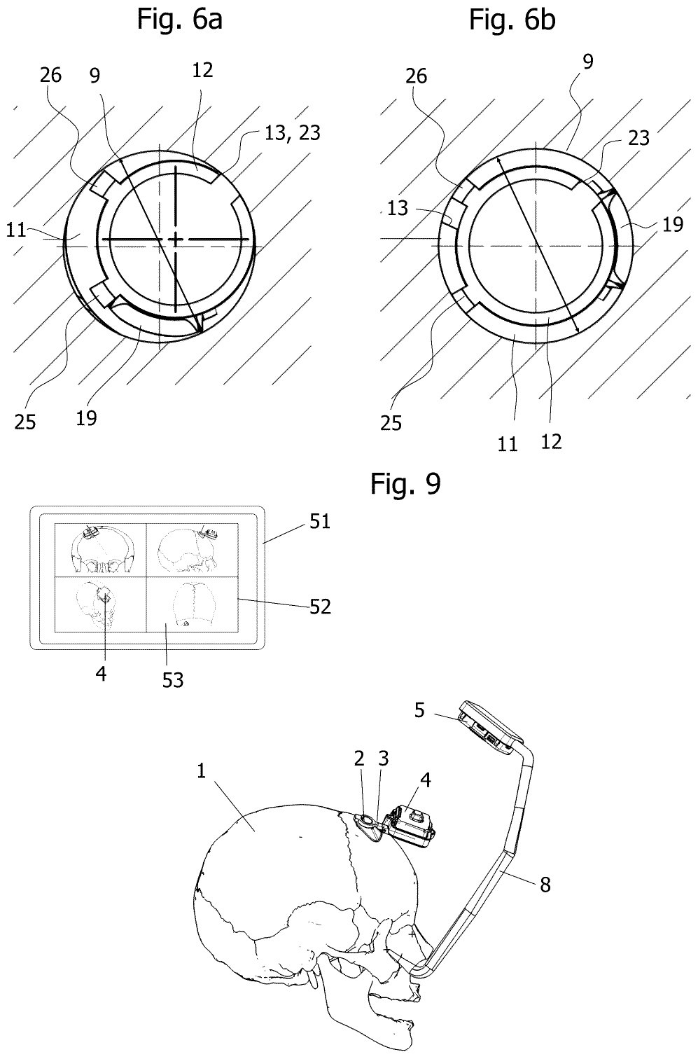

FIG. 2b shows the fixation device 2 in the assembled state. The first sleeve element portion 11 contains a grip element 18, which is firmly connected to the first sleeve element portion 11. In other words, by operating the grip element 18, a rotation of the first sleeve element portion 11 relative to the second sleeve element portion 12 is triggered. The grip element 18 is configured in particular for manual operation. According to the present embodiment, the grip element 18 can be moved between the first position shown in FIG. 2a, FIG. 2b, FIG. 3a, FIG. 4a, FIG. 4b, FIG. 6a and a second position shown in FIG. 3b, FIG. 5a, FIG. 5b, FIG. 6b. In the illustration shown in FIG. 2b, the fixation device 2 is shown in the installed state. In the installed state, the fixation device 2 is positioned in the opening 9. The opening 9 is omitted in the present illustration in order to provide a view of the components located inside the opening 9.

The fixation device 2 shown in FIG. 2b is shown in its first position, in which the installation of the fixation device 2 in the opening 9 or the removal of the fixation device 2 from the opening 9 is possible. During insertion, the sleeve element 10 is positioned in the opening 9 in such a way that the flange 16 or the grip element 18 rests on the outer surface of the body part containing the opening 9, in particular a layer of tissue covering a skull bone 1. In order to facilitate the introduction of the sleeve element 10 into the opening 9, a clamping force can be exerted on the casing parts of the first and second sleeve element portions 11, 12 located on either side of the longitudinal slots 13, 23, which causes the slot width of the longitudinal slots 13, 23 to be reduced. In the first position shown in FIG. 2b, the longitudinal slots 13, 23 are aligned so that a particularly energy-efficient reduction of the cross-sectional area of the sleeve element 10 can be achieved.

In the first position shown, the first and second projections 25, 26 of the second sleeve element portion 12 are adjacent to or near the extension 19 of the first sleeve element portion 11, which is particularly visible in FIG. 4b. The projections 25, 26 and the extension 19 form an angle of no more than 200 degrees, in particular no more than 180 degrees, in a plane perpendicular to the longitudinal axis. In the first position, the cross-sectional area of the second sleeve element portion 12 with the first and second projections 25, 26 and the extension 19 of the first sleeve element portion 11 is thus at most equal to the cross-sectional area of the flange 17 of the first sleeve element portion 11. In the first position, the fixation element 2 can thus be introduced into the opening 9, since the opening 9 has a cross-sectional area that is somewhat larger than the cross-sectional area of the flange 17 of the first sleeve element portion 11.

FIG. 3a shows an exploded view of the fixation device 2 without the tracker, where the view according to FIG. 3a is offset at an angle of about 20 to 40 degrees with respect to the view according to FIG. 2a. The fixation device 2 is also shown in the first position in FIG. 3a. In FIG. 3a it is more clearly visible that the longitudinal slot 13 of the first sleeve element portion 11 is aligned with the longitudinal slot 23 of the second sleeve element portion 12. In addition, the first projection 25 and the second projection 26 of the second sleeve element portion 12 are visible.

FIG. 3b is a view of the fixation device 2 with a holding device 31 for a tracker in the second position. In the second position, the fixation device 2 is received and fixed in the opening 9. The opening 9 is omitted in the present illustration in order to better clarify the position of the components. The second position is obtained from the first position shown in FIG. 2b by fixing the fixation device 2 located in the opening 9 as shown in FIG. 2b. The fixation device 2 is fixed in the opening by operating the grip element 18, which is turned by a maximum of 90 degrees about the common longitudinal axis of the sleeve element 10 compared to the position shown in FIG. 2b. The grip element 18 is firmly connected to the first sleeve element portion 11, so that when the grip element 18 is turned, the first sleeve element portion 11 can be moved relative to the second sleeve element portion 12.

In FIG. 3b, the longitudinal slot 23 of the second sleeve element portion 12 is still visible, but the longitudinal slot 13 of the first sleeve element portion 11 is no longer visible. When the first sleeve element portion 11 is rotated relative to the second sleeve element portion 12, the position of the extension 19 connected to the first sleeve element portion 11 also changes. In the second position, the extension 19 is in an opposite position with respect to the first and second projections 25, 26 of the second sleeve element portion 12. In the second position, both the first and second projections 25, 26 and the extension 19 are in contact with the inner wall of the opening 9. In the second position, the fixation device 2 is fastened in the opening 9 in such a way that slipping of the holding element 3 and of a holding device 31 fastened to the holding element 3 is prevented. In particular, a profiling 20 located on the outer surface of the extension 19 can serve to further increase the frictional resistance. If the profiling 20 contains sharp-edged elements, for example ribs, grooves, teeth, spikes, points, cutting elements or threads, the profiling 20 can even engage with the inner wall of the opening 9, so that a firm fit of the fixation device 2 in the opening 9 can be ensured. The flange 17 of the first sleeve element portion 11 can be used to improve centering and support of the fixation device 2 during installation of the same in the opening 9.

FIG. 4a shows a perspective view of the fixation device 2 and the holding element 3 in the first position. The holding element 3 is firmly connected to the second sleeve element portion 12. In the present embodiment, the first end 21 of the second sleeve element portion 12 forms a common surface with the holding element 3, ideally, this connection is configured to be gap-free so that the fixation device 2 can be easily cleaned. Of course, the second sleeve element portion 12 and the holding element 3 can also be designed as a single component. The first sleeve element portion 11 can be pushed over the second sleeve element portion 12 until it rests against the underside of the holding element 3. According to the present embodiment, the flange 16, which is arranged at the first end 14 of the first sleeve element portion 11, has a larger outer diameter than the holding element 3, so that the flange 16 mainly extends beyond the holding element 3. The holding element 3 is formed as a rod-shaped body with an annular extension, which is configured to be received on the second sleeve element portion 12. The annular extension contains a thickening, which, according to the present example, is arranged opposite the rod-shaped body. This thickening serves as a stop for the grip element 18.

FIG. 4b shows a perspective view of the fixation device 2 and the holding element 3 in the first position, in which view the second end 22 of the second sleeve element portion 12 and the second end 15 of the first sleeve element portion 11 are visible. Also shown in FIG. 4b are the extension 19 of the first sleeve element portion 11 and the first projection 25 and the second projection 26 of the second sleeve element portion 12. FIG. 4b also clearly shows that in the first position, the first and second projections 25, 26 and the extension 19 occupy approximately half the circumference of the second sleeve element portion 12. In this first position, the fixation device 2 thus has a minimal cross-sectional area, which makes it particularly easy to insert it into the opening 9. In addition, it is possible to use a tool to reduce the width of the longitudinal slot 13 of the first sleeve element portion 11 and the longitudinal slot 23 of the second sleeve element portion 12, so that the fixation element 2 can be more easily inserted into the opening 9.

FIG. 5a shows a perspective view of the fixation device 2 and the holding element 3 in the second position, according to which the first sleeve element portion 11 has been twisted through an angle of approximately 90 degrees relative to the second sleeve element portion 12. The grip element 18 extends opposite the rod-shaped body of the holding element 3.

FIG. 5b shows a perspective view of the fixation device 2 and the holding element 3 in the second position, in which view the second end 22 of the second sleeve element portion 12 and the second end 15 of the first sleeve element portion 11 are visible. FIG. 5b also shows the extension 19 of the first sleeve element portion 11 and the first projection 25 and the second projection 26 of the second sleeve element portion 12. It can also be clearly seen in FIG. 5b that in the second position the first and second projections 25, 26 and the extension 19 are arranged approximately opposite each other. The arrangement of the projections 25, 26 and the extension 19 is such that they exert a clamping effect, so that the fixation device 2 is fixed in the opening 9. In this second position, the fixation device 2 thus has a maximum cross-sectional area, which ensures an optimal hold in the opening 9. In addition, it is possible that the extension 19 has a profiling that engages with the inner wall of the opening 9, so that the fixation device is locked in the second position. The first and second projections can also have a profiling by means of which at least the frictional resistance on the inner wall of the opening can be increased. In particular, the profiling of the first and second projections 25, 26 can be configured in such a way that they can engage in the inner wall of the opening 9. The position of the first sleeve element portion 11 relative to the second sleeve element portion 12 in the second position depends on the inner diameter of the opening 9. The opening 9 can have an effective diameter that differs slightly from the nominal diameter. For example, if the nominal diameter of the opening 9 is in the range of 10 to 15 mm inclusive, the effective diameter can deviate from the nominal diameter by up to 1 mm. The position of the first sleeve element portion 11 in the second position is adjustable so that deviations between the nominal diameter and the actual diameter of the opening 9 can be compensated by the fixation device. The position of the extension 19 relative to the first and second projections 25, 26 can thus be set differently in the second position depending on the effective diameter of the opening 9.

FIG. 6a shows a radial section through the fixation device 2 according to FIG. 2a in the first position, which is inserted into an opening 9 shown in section. The fixation device 2 is advantageously not inserted coaxially with the center axis of the opening 9, but at an angle of inclination, so that the extension 19 of the first sleeve element portion 11 and the projections 25 and 26 of the second sleeve element portion 12 do not come into contact with the inner wall of the opening 9 when the fixation device 2 is inserted into the opening 9.

FIG. 6b shows a radial section through the fixation device 2 according to FIG. 3b in the second position, which is located in the opening 9.

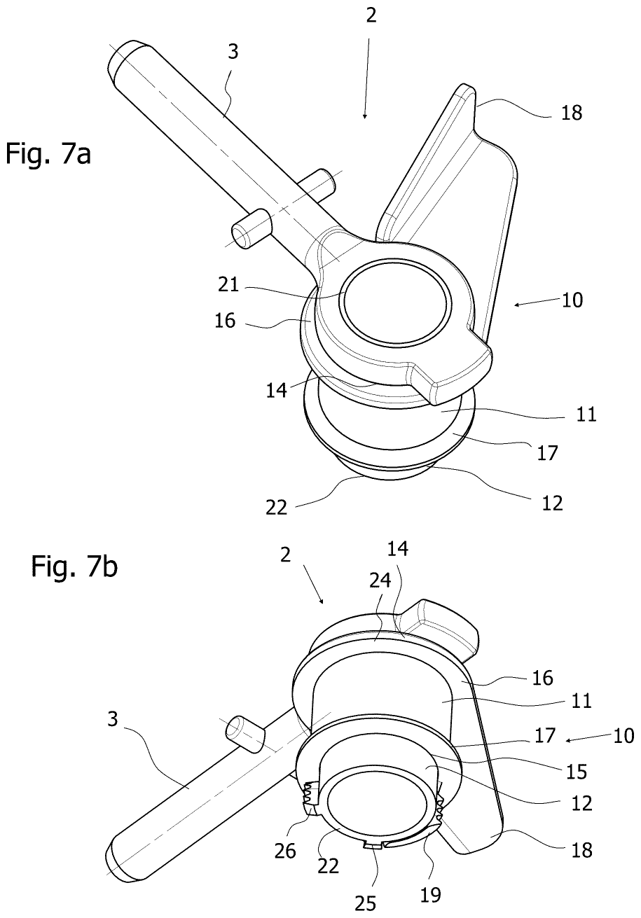

FIG. 7a shows a perspective view of a fixation device 2 according to a second embodiment with the holding element 3 in a first position. For this embodiment, the same reference signs are used for identical or equivalent components as for the first embodiment. According to the second embodiment, the first sleeve element portion 11 and the second sleeve element portion 12 do not have a longitudinal slot. The holding element 3 is firmly connected to the second sleeve element portion 12. In the present embodiment, the first end 21 of the second sleeve element portion 12 forms a common surface with the holding element 3, ideally this connection is configured to be gap-free so that the fixation device 2 can be easily cleaned. Of course, the second sleeve element portion 12 and the holding element 3 can also be designed as a single component. The first sleeve element portion 11 can be pushed over the second sleeve element portion 12 until it rests against the underside of the holding element 3. According to the present embodiment, the flange 16, which is at the first end 14 of the first sleeve element portion 11, has a larger outer diameter than the holding element 3, so that the flange 16 mostly protrudes over the holding element 3. The holding element 3 is formed as a rod-shaped body with an annular extension, which is configured to be received on the second sleeve element portion 12. The annular extension contains a thickening, which, according to the present example, is arranged opposite the rod-shaped body. This thickening serves as a stop for the grip element 18.

FIG. 7b shows a further perspective view of the fixation device according to FIG. 7a with the holding element 3 in the first position, wherein in this view the second end 22 of the second sleeve element portion 12 and the second end 15 of the first sleeve element portion 11 are visible. FIG. 7b also shows the extension 19 of the first sleeve element portion 11 and the first projection 25 and the second projection 26 of the second sleeve element portion 12. It can also be clearly seen in FIG. 7b that in the first position, the first and second projections 25, 26 and the extension 19 occupy approximately half of the circumference of the second sleeve element portion 12. In this first position, the fixation device 2 thus has a minimal cross-sectional area, which makes its insertion into the opening 9 particularly easy.

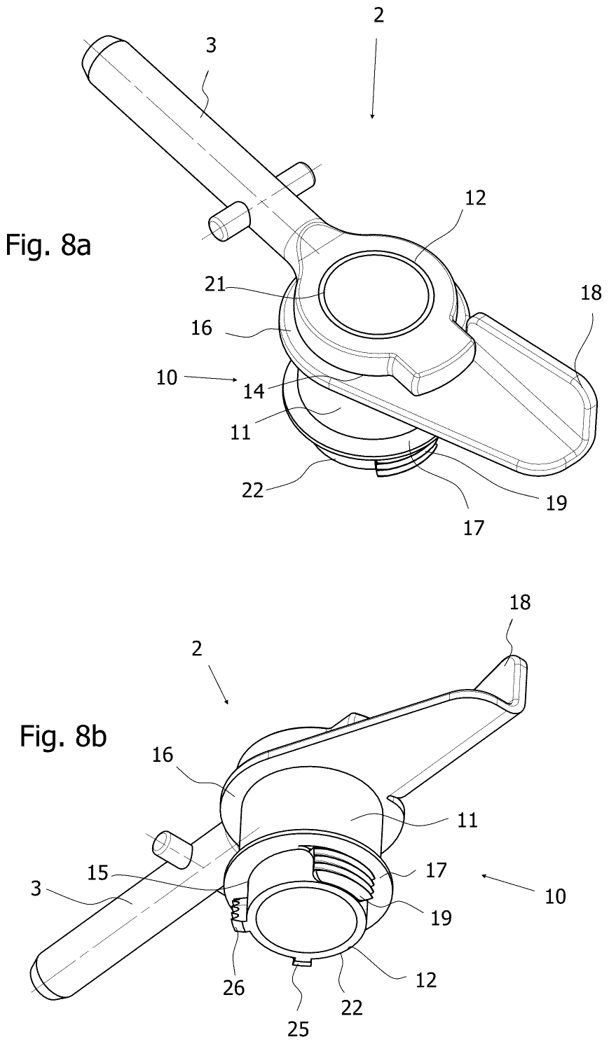

FIG. 8a shows a perspective view of the fixation device 2 according to the second embodiment with the holding element 3 in the second position, according to which the first sleeve element portion 11 has been twisted through an angle of approximately 90 degrees relative to the second sleeve element portion 12. The grip element 18 extends opposite the rod-shaped body of the holding element 3.

FIG. 8b shows a further perspective view of the fixation device 2 with the holding element 3 in the second position, in which view the second end 22 of the second sleeve element portion 12 and the second end 15 of the first sleeve element portion 11 are visible. FIG. 8b also shows the extension 19 of the first sleeve element portion 11 and the first projection 25 and the second projection 26 of the second sleeve element portion 12. It can also be clearly seen in FIG. 8b that in the second position, the first and second projections 25, 26 and the extension 19 are arranged approximately opposite each other. The projections 25, 26 and the extension 19 are arranged in such a way that they exert a clamping effect, so that the fixation device 2 is fixed in the opening 9. In this second position, the fixation device 2 thus has a maximum cross-sectional area, which ensures an optimal hold in the opening 9. In addition, it is possible for the extension 19 to have a profiling that engages with the inner wall of the opening 9, so that the fixation device is locked in the second position. The first and second projections can also have a profiling by means of which at least the frictional resistance on the inner wall of the opening can be increased.

In particular, the profiling of the first and second projections 25, 26 can be designed such that they can engage in the inner wall of the opening 9. The position of the first sleeve element portion 11 relative to the second sleeve element portion 12 in the second position is dependent on the internal diameter of the opening 9. The opening 9 may have an effective diameter that differs slightly from the nominal diameter. For example, if the nominal diameter of the opening 9 is in the range of 10 to 15 mm inclusive, the effective diameter can deviate from the nominal diameter by up to 1 mm. The position of the first sleeve element portion 11 in the second position is adjustable so that deviations between the nominal diameter and the effective diameter of the opening 9 can be compensated by the fixation device 2. The position of the extension 19 relative to the first and second projections 25, 26 can thus be adjusted differently in the second position depending on the effective diameter of the opening 9.

FIG. 9 shows an example of the use of the fixation device according to one of the embodiments. According to the present embodiment, the fixation device is used for a navigation system. The fastening device contains the holding element 3 and a first tracker 4, which is configured, for example, as an optical marker. A therapy device 6, in accordance with the present example a pointer 8, is equipped with a second tracker 5. The second tracker 5 shown in FIG. 9 contains a single camera configured for an application of shadow imaging technology. According to the present embodiment, a first optical marker attached to the fixation device 2 can be detected by means of the navigation system. In particular, 6DOF information can be obtained regarding the position of the optical marker in space and/or the relative position of the first optical marker and the second tracker 5, which may contain a second optical marker. A display unit 52 can be used to register the patient's anatomy from a preoperative scan to the first tracker 4 attached to the bone by the fixation device 2 before using a therapy device 6 that includes the second tracker 5 for the navigation system.

The navigation system includes a computing unit 51 and a display unit 52 for calculating the position of the first tracker 4 based on tracking data. The display unit 52 can display 3D models of the anatomy and the tracker 4 or, if preoperative images are loaded, the position of the tracker 4 in one or more image slices 53. The computing unit 51 can be integrated into the display unit 52 or can be separate from it. The display unit 51 can, for example, be configured as a monitor, a head-up display or an augmented reality display. The navigation system can be connected to the computing unit 51 by a cable. Alternatively, tracking data can be transmitted wirelessly from a second tracker 5, for example a camera, to the computing unit 51.

It is obvious to a person skilled in the art that many further variants in addition to the systems or process variants described are possible without deviating from the inventive concept. The subject matter of the invention is thus not limited by the preceding description and is determined by the scope of protection defined by the claims. For the interpretation of the claims or the description, the broadest possible reading of the claims is decisive. In particular, the terms “comprising” or “including” are to be interpreted as referring to elements, components or steps in a non-exclusive meaning, thereby indicating that the elements, components or steps may be present or used, that they may be combined with other elements, components or steps not explicitly mentioned. When the claims refer to an element or component from a group which may consist of A, B, C to N elements or components, this formulation is to be interpreted as requiring only one element of this group, and not a combination of A and N, B and N or any other combination of two or more elements or components of this group.

What is claimed is:

1. A fixation device for fastening a therapy device in an opening arranged in a bone, comprising a sleeve element which is configured to be received in the opening, wherein the sleeve element comprises a first sleeve element portion and a second sleeve element portion, wherein the first sleeve element portion surrounds the second sleeve element portion in an installed state, wherein the first sleeve element portion is configured as a cylindrical hollow body with a longitudinal axis, wherein the first sleeve element portion has a first end and a second end, wherein the first end of the first sleeve element portion includes a grip element, wherein the second end of the first sleeve element portion includes an extension, wherein the second sleeve element portion is configured as a cylindrical hollow body having a longitudinal axis, wherein the second sleeve element portion contains a projection arrangement, wherein the first sleeve element portion and the second sleeve element portion are rotatable about one another in such a way that the projection arrangement is arranged in a first position adjacent to the extension and the projection arrangement is arranged at a distance from the extension in a second position.

2. The fixation device of claim 1, wherein the first sleeve element portion includes a first flange and a second flange.

3. The fixation device of claim 2, wherein the extension projects beyond the second flange in a direction of the longitudinal axis of the cylindrical hollow body of the first sleeve element portion.

4. The fixation device of claim 1, wherein the extension extends over a part of a circumference of the first sleeve element portion.

5. The fixation device of claim 1, wherein the extension has a segment length which is in a range of from 10% to 40% inclusive of a circumference of the cylindrical hollow body of the first sleeve element portion.

6. The fixation device of claim 1, wherein the extension has an axial length that is in a range of from 5% to 50% inclusive of the distance measured between the first end and the second end in a direction of the longitudinal axis of the first sleeve element portion.

7. The fixation device of claim 1, wherein the extension has a profiling on the outside.

8. The fixation device of claim 1, wherein the second sleeve element portion has an outside which includes at least one recess.

9. The fixation device of claim 1, wherein the second sleeve element portion includes a holding element.

10. The fixation device of claim 1, wherein the projection arrangement includes a first projection and a second projection, wherein the projection arrangement can include a profiling.

11. The fixation device of claim 1, wherein the second sleeve element portion has an inside diameter amounting to at least 3 mm.

12. The fixation device of claim 1, wherein the first sleeve element portion contains a longitudinal slot.

13. The fixation device of claim 12, wherein the longitudinal slot extends from the first end of the first sleeve element portion to the second end of the first sleeve element portion.

14. The fixation device of claim 1, wherein the second sleeve element portion contains a longitudinal slot.

15. The fixation device of claim 14, wherein the longitudinal slot extends from a first end of the second sleeve element portion to a second end of the second sleeve element portion.

16. A navigation system, comprising a fixation device for fastening a therapy device in an opening arranged in a bone, wherein the fixation device comprises a sleeve element which is configured to be received in the opening, wherein the sleeve element comprises a first sleeve element portion and a second sleeve element portion, wherein the first sleeve element portion surrounds the second sleeve element portion in an installed state, wherein the first sleeve element portion is configured as a cylindrical hollow body with a longitudinal axis, wherein the first sleeve element portion has a first end and a second end, wherein the first end of the first sleeve element portion includes a grip element, wherein the second end of the first sleeve element portion includes an extension, wherein the second sleeve element portion is configured as a cylindrical hollow body having a longitudinal axis, wherein the second sleeve element portion contains a projection arrangement, wherein the first sleeve element portion and the second sleeve element portion are rotatable about one another in such a way that the projection arrangement is arranged in a first position adjacent to the extension and the projection arrangement is arranged at a distance from the extension in a second position.

17. The navigation system of claim 16, wherein the fixation device is configured to fasten a first tracker in the opening arranged in the bone, wherein the navigation system comprises a therapy device which includes a second tracker, a tracking system, a computing unit and a display unit, wherein the tracking system is configured to determine a position of the therapy device relative to at least one of the first tracker or the second tracker, wherein the computing unit is configured to calculate the position of the therapy device in an anatomical coordinate frame, wherein the display unit is configured to display a virtual position of the therapy device with respect to registered preoperative images or 3D models.
